(19) United States
(12) Patent Application Publication
(10) Pub. No.: US 2022/0133297 A1
(43) Pub. Date: May 5, 2022

(54) SHUTTLE CABLE TRANSPORTING DEVICE AND METHOD

(71) Applicant: Mason James Bettenga, Memphis, TN (US)

(72) Inventor: Mason James Bettenga, Memphis, TN (US)

(21) Appl. No.: 17/090,707

(22) Filed: Nov. 5, 2020

(51) Int. Cl. A61B 17/04

(52) U.S. Cl. CPC A61B 17/0491; A61B 2017/564; A61B 17/0483

(57) ABSTRACT

A surgical device for controlling a shuttle cable, comprises a shaft, a capture mechanism comprising a first and second jaw and cavity therebetween accessible by a shuttle cable, and a gate connected to a jaw and in rigid communication with a flexible hinge, the hinge operable to allow the gate to deflect from a closed position to an open position by a force acting on the distal side of the gate, wherein the gate resiliently returns from an open position to a closed position, the capture mechanism is operable to allow the shuttle cable to slide axially through a containment aperture. A method comprises guiding the surgical device through a bone aperture, coupling the surgical device to a portion of the shuttle cable; and retrieving the capture mechanism through the aperture to the first side of the bone.

### CROSS REFERENCE TO RELATED APPLICATIONS

This application claims priority to U.S. Provisional App. No. 62/931,375 filed Nov. 6, 2019, titled SUTURE TRANSPORTING DEVICE AND METHOD, herein incorporated by reference in its entirety for all purposes.

### FIELD OF THE INVENTION

The present invention relates to devices and methods for orthopedic tissue reconstruction procedures requiring transporting a surgical device through bone tissue.

### BACKGROUND

In the field of orthopedic surgery, several different instruments have been developed for retrieving a flexible cable through tissue to be used as a shuttling device for the purpose of transferring an instrument, an implant, or tissue from one location to another inside a surgical site. However, the limitation with the current marketed devices is the reliability of the coupling method. For example, needles having a crochet-style hook formed into the exterior surface may be used to catch a loop of the cable and passed through tissue, however, such devices do not provide a means for securing the cable and may allow disengagement from the retrieval device, particularly if the user requires the shuttle cable to slide in the hook during the retrieval process. Multiple retrieval attempts may be necessary which can be a source of unnecessary delay during a surgical procedure. Therefore, a clear need exists for a suture transporting device which solves the aforementioned problem.

### BRIEF SUMMARY OF THE INVENTION

In one embodiment of the invention a surgical device for controlling a shuttle cable is provided, comprising a shaft, comprising an elongate member having a longitudinal axis between a proximal end and a distal end, and a capture mechanism formed at the distal end of said shaft, comprising a mouth, comprising a first jaw and a second jaw, the first and second jaws having cavity therebetween and accessible by a shuttle cable, the shuttle cable comprising a flexible member having a first end and a second end and a portion therebetween, and a gate comprising at least one elongate body having a first end and a second end and a proximal side and a distal side, the first end connected to a jaw and in rigid communication with a flexible hinge, the hinge operable to allow the gate to deflect from a closed position to an open position by a force acting on the distal side of the gate, wherein the gate resiliently returns from an open position to a closed position in the absence of said opening force, and a containment aperture, the aperture bounded by the proximal side of the gate, the first jaw, the second jaw, and the body of the capture mechanism and operable to allow the shuttle cable to slide axially therethrough, wherein the open position of the gate allows passage of a portion of the shuttle cable into the containment aperture and the closed position of the gate separates the mouth from the containment aperture.

Also in one embodiment, the surgical device further comprises a means for orienting the shaft to a feature on a second device about the longitudinal axis of the shaft, the means comprising at least one of the following: (a) a boss engaging a slot on the second device, (b) a slot engaging a boss on the second device, (c) a feature formed on the device compared to a feature on a second device, wherein the capture mechanism is operable for a shuttle cable coupled with the second device to pass through the gate when said features are aligned.

In another aspect of the invention, the surgical device for controlling a shuttle cable comprises a shaft and a capture mechanism, comprising a tubular body and a helical aperture formed radially from the lumen to the outer surface, the aperture connecting the distal surface to the proximal end of a containment aperture and operable for the passage of a shuttle cable, the shuttle cable comprising a flexible member having a first end and a second end and a portion therebetween and a containment aperture extending radially from the lumen to the outer surface and formed at the proximal end of the helical aperture, wherein the containment aperture is operable to allow a shuttle cable to slide therethrough, and rotation of the surgical device about the longitudinal axis causes the shuttle cable to enter the helical aperture, and rotation of the suture transporting device causes a portion of the shuttle cable to translate proximally, and rotation of the surgical device causes a portion of the shuttle cable to enter the containment aperture.

In another embodiment of the invention, a method of transporting a shuttle cable through tissue is presented, comprising the steps: (a) guiding the surgical device from a first side of a bone to a second side through an aperture formed therethrough, (b) aligning the capture mechanism of the surgical device with a portion of a shuttle cable, operable for engagement with the capture mechanism, and a portion of the flexible member spans a portion of the bone aperture on the second side of the bone aperture, (c) coupling the surgical device to a portion of the shuttle cable, and (d) retrieving the capture mechanism through the bone aperture to the first side of the bone, wherein the flexible member is operable to slide through the containment aperture, and the first end of the shuttle cable is available on the first side of the bone and the second end of the shuttle cable remains on the second side of the bone, and a portion of the shuttle cable is contained in the bone aperture.

In another embodiment, the aforementioned surgical device further comprises a handle rigidly coupled to the proximal end of the shaft. Also in one embodiment, the handle further comprises a plurality of gripping features to provide increased traction during grasping and manipulation of the transporting device during use. Also in one embodiment, the orienting means is connected to a handle.

### BRIEF DESCRIPTION OF THE DRAWINGS

The foregoing and other objects, features, and advantages will be apparent from the following more elaborate description of the embodiments, as illustrated in the accompanying drawings in which like reference characters refer to the same parts throughout the different views. The drawings are not necessarily to scale, emphasis instead being placed upon illustrating the principles of the embodiments. For a detailed description of example embodiments, reference will now be made to the accompanying drawings in which:

FIG. 1 illustrates perspective and detail views of a shuttle cable transporting device, according to an embodiment of the present invention;

FIGS. 2A-2B are front, side and perspective views illustrating the capture mechanism of FIG. 1, in accordance with the disclosure;

FIG. 3 illustrates the operation of the capture mechanism of FIG. 1, in accordance with the disclosure;

FIGS. 4A-4B show one example of a drill guide system that may be used with the shuttle cable transporting device of FIG. 1, in accordance with the disclosure;

FIGS. 5A-5C illustrate exemplary steps for capturing a shuttle cable integrated onto the drill guide of FIGS. 4A-4B using the shuttle cable transporting device of FIG. 1, in accordance with the disclosure;

FIGS. 6A-6C illustrate exemplary steps of employing the shuttle cable transporting device of FIG. 1 to retrieve a shuttle cable through a prepared bone aperture, according to an embodiment of the present invention;

FIG. 7 illustrates perspective and detail views of a shuttle cable transporting device, according to an alternative embodiment of the present invention;

FIGS. 8A-8B illustrate the operation of the capture mechanism of the shuttle cable transporting device of FIG. 7, in accordance with the disclosure;

FIG. 9 illustrates perspective and detail views of a shuttle cable transporting device, according to an alternative embodiment of the present invention;

FIGS. 10A-10B illustrate the operation of the capture mechanism of the suture transporting device of FIG. 9, in accordance with the disclosure;

FIG. 11 illustrates perspective and detail views of a shuttle cable transporting device, according to an alternative embodiment of the present invention;

FIGS. 12A-12C illustrate the operation of the capture mechanism of the suture transporting device of FIG. 11, in accordance with the disclosure.

While the invention is amenable to various modifications, permutations, and alternative forms, specific embodiments have been shown by way of example in the drawings and are described in detail below. The intention, however, is not to limit the invention to the embodiments described. The invention is intended to cover all modifications, equivalents, and alternatives falling within the scope of the invention as defined by the appended claims.

### DETAILED DESCRIPTION

The inventor provides a unique suture transporting device for safely and reliably retrieving a shuttle cable through tissue during surgical reconstruction procedures. The present invention is described in enabling detail in the following examples, which may represent more than one embodiment of the present invention. Although one or more of these embodiments may be preferred, the embodiments disclosed should not be interpreted, or otherwise used, as limiting the scope of the disclosure, including the claims. In addition, one skilled in the art will understand that the following description has broad application in several surgical scenarios with various anatomical structures, and the discussion of any embodiment is meant only to be exemplary of that embodiment, and not intended to intimate that the scope of the disclosure, including the claims, is limited to that embodiment. It should be understood that for the purposes of this discussion, the nomenclature of shuttle cable should be interpreted for broad application and refer to any suture, cable, braid, wire, tape, or other flexible member operable for surgical application.

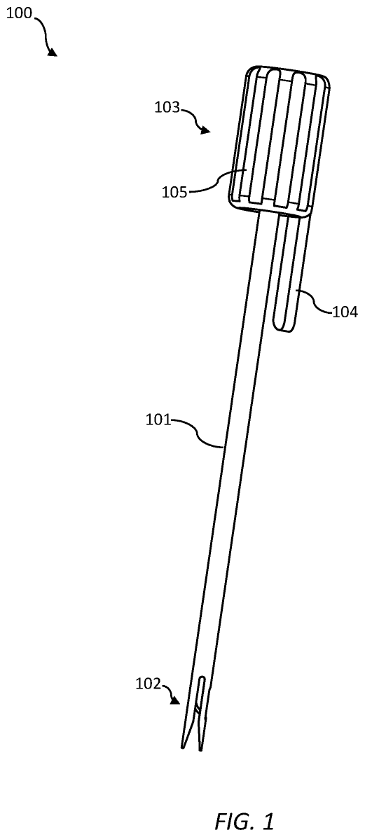

Referring now to FIG. 1, one embodiment of a shuttle cable transporting device 100 is shown in a perspective view and comprises a shaft 101, a capture mechanism 102, and a handle 103. The components of shuttle cable transporting device 100 may be manufactured using standard machining techniques, laser cutting, wire EDM, moulding processes, casting processes, additive manufacturing or other methods using bio-compatible materials suitable for surgical instrumentation. These materials include, but are not limited to alloys of stainless steel, alloys of titanium, thermoplastics such as polyphenyl sulfone (PPSU), polyoxymethylene (acetal), polyether-etherketone (PEEK), or fiber-reinforced composites using thermoset or thermoplastic resins. Shaft 101 may be a solid or tubular body having an outer diameter ranging from approximately 2 millimeters (mm) to 10 mm having a long axis between a proximal end and a distal end and having a length operable to span anatomical structures. Capture mechanism 102 is located at the distal end of shaft 101 and may be affixed as a separate component or integrated into shaft 101 as a single component. Handle 103 is an elongate body having a long axis between a proximal end and a distal end and may be manufactured as a separate component and affixed the proximal end of shaft 101 or integrated with shaft 101 as a single component. Extending distally from the outer edge of the distal end of handle 103 is an orienting arm 104 operable to engage an orienting slot in a surgical guide. Handle 103 is affixed on shaft 101 such that the engagement of orienting arm 104 with a calibrated slot in a surgical guide will provide capture mechanism 102 oriented to enable the coupling of a shuttle suture for retrieval. In this embodiment, handle 103 further comprises a plurality of traction grooves 105 intended to provide the user additional grip for during manipulation of the device.

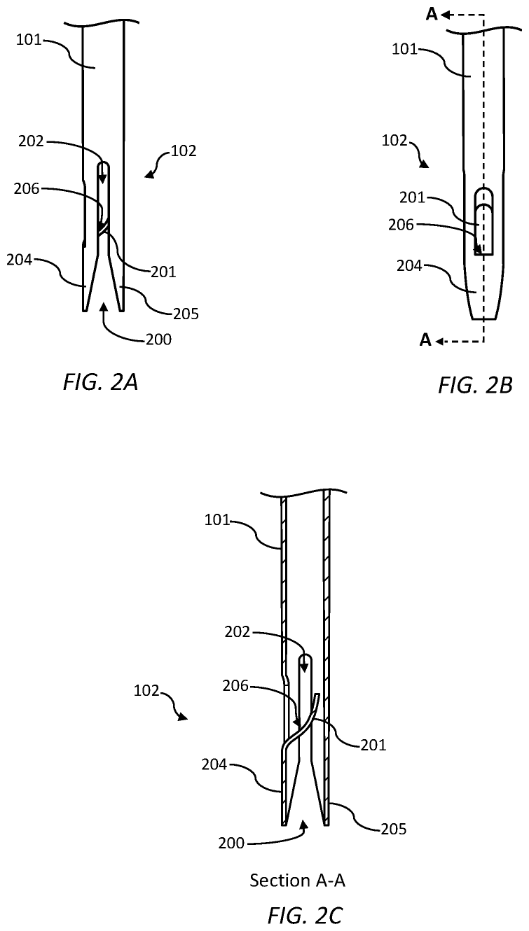

FIGS. 2A-2B are front and side views, respectively, illustrating detailed views of capture mechanism 102, according to an embodiment of the present invention. In this example, shaft 101 is a tubular body having a longitudinal axis between a proximal end and a distal end, incorporating capture mechanism 102 at the distal end. A mouth 200 is formed between a first jaw 204 and a second jaw 205 at the distal end of capture mechanism 102, and is operable for guiding a shuttle suture between jaws 204 and 205 toward a gate 201 which has a generally flat body having a proximal side and a distal side, and is connected to jaw 204 by a flexible hinge 203. Gate 201 extends proximally, spanning mouth 200 at an angle, and is enabled to open proximally and away from jaw 205 when a moment is applied to the distal side of gate 201 and resiliently close toward jaw 205 when the opening force is released, allowing a shuttle suture access to a cable containment aperture 202 and be retained in said aperture. Thus, capture mechanism 102 forms a one-way valve for allowing a shuttle cable presented between jaws 204 and 205 to open and pass by gate 201 in a proximal direction, and is subsequently prevented from translation in the distal direction by the closed gate 201. Cable containment aperture 202 is formed having dimensions sufficient for allowing the shuttle suture to slide freely on a saddle 206. FIG. 2C is a section view of capture mechanism 102 further illustrating the orientation of gate 201 to mouth 200, cable containment aperture 202, and saddle 206.

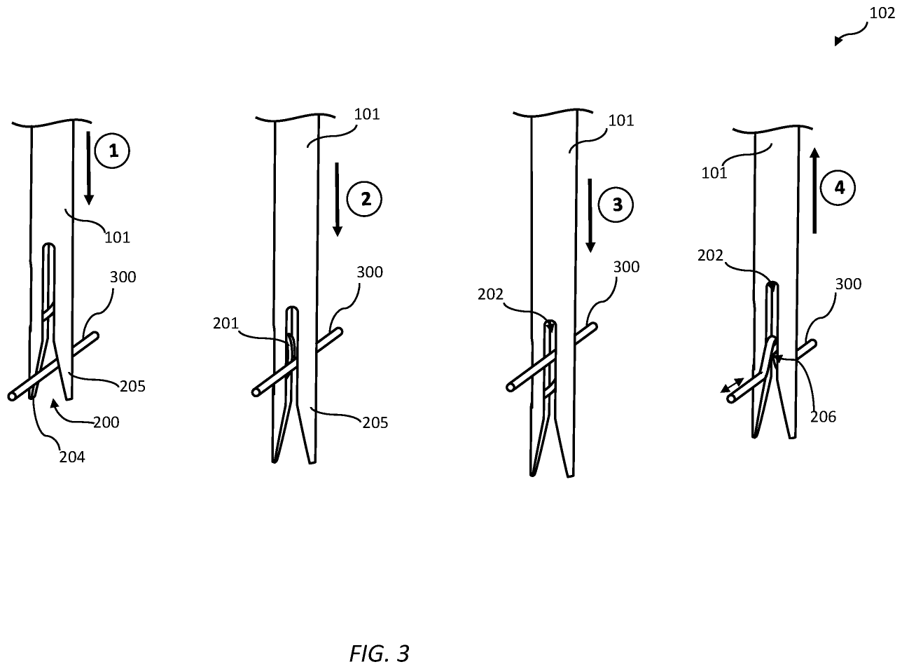

FIG. 3 illustrates exemplary steps for employing capture mechanism 102 to couple with a shuttle cable 300, in accordance with the disclosure. In a first step, shuttle cable 300 is approximated generally perpendicular to mouth 200 between jaws 204 and 205 as shaft 101 is translated distally along its longitudinal axis toward shuttle cable 300. In a second step, shuttle cable 300 contacts the distal side of gate 201 causing it to flexibly open away from jaw 205. It should be noted that a necessary condition to cause gate 201 to open is shuttle cable 300 being held sufficiently taut. In a third step, shuttle cable 300 is translated proximally past gate 201 by further advancing shaft 101 in a distal direction. In this position, gate 201 resiliently closes capturing shuttle cable 300 in cable containment aperture 202 where it may axially slide on saddle 206. A fourth step is illustrated as the retrieval of shuttle cable 300 by translating shaft 101 in a proximal direction with shuttle cable 300 retained.

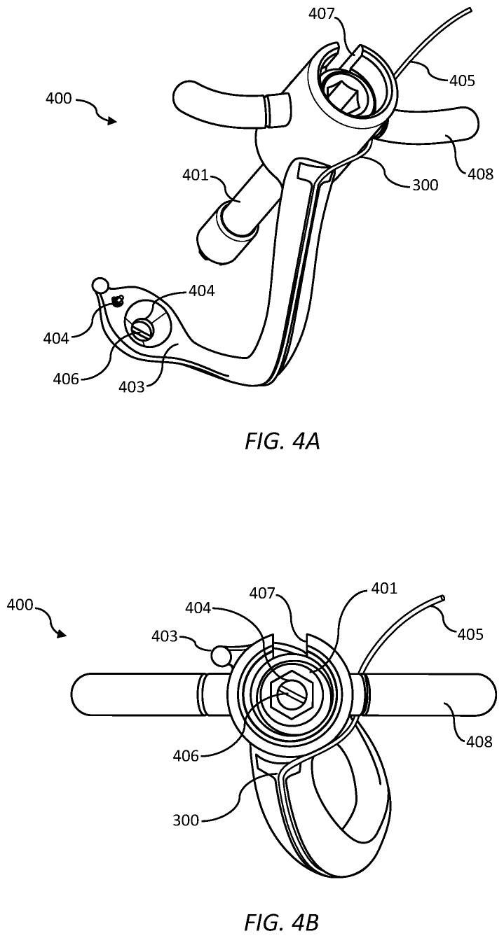

FIGS. 4A-4B illustrate one example of a drill guide system 400 that may be used with shuttle cable transporting device 100, in accordance with the disclosure. Drill guide system 400 has an aperture 403 formed in a foot 402 and is oriented colinear with the inner diameter of a guide sleeve 401 operable to direct capture mechanism 102 into aperture 403 where a portion 406 of shuttle cable 300 is provided. In this example, a first end 404 of shuttle cable 300 is coupled to foot 402 whilst a second end 405 is coupled to an arm 408 such that portion 406 spans aperture 403 having the necessary tension for engagement with capture mechanism 102. An orienting slot 407 is formed in drill guide system 400 such that engagement with orienting arm 104 will align jaws 204 and 205 to receive shuttle cable 300.

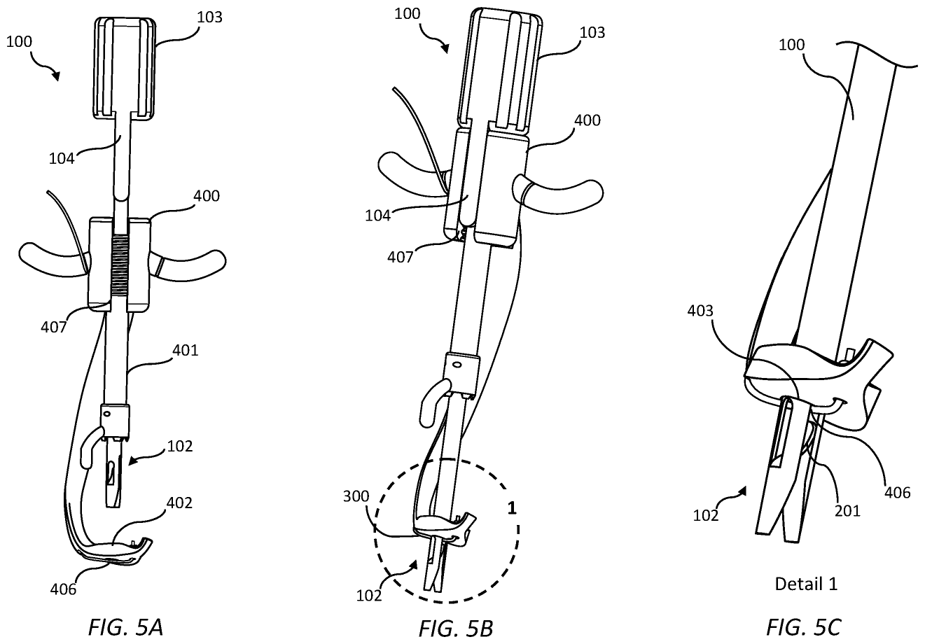

FIGS. 5A-5C illustrate exemplary steps for capturing shuttle cable 300 affixed to drill guide system 400 using shuttle cable transporting device 100, according to one embodiment of the present invention. As shown in FIG. 5A, shuttle cable transporting device 100 is passed into guide sleeve 401 and translated distally towards foot 402. Shuttle cable transporting device 100 is rotated about is long axis such that orienting arm 104 is aligned with orienting slot 407. In this orientation, capture mechanism 102 is rotationally aligned to engage portion 406. FIG. 5B illustrates shuttle cable transporting device 100 further advanced distally through aperture 403 until portion 406 has passed through gate 201 and is now retained by capture mechanism 102. FIG. 5C shows a detail view illustrating the elements referenced in the description of FIG. 5B.

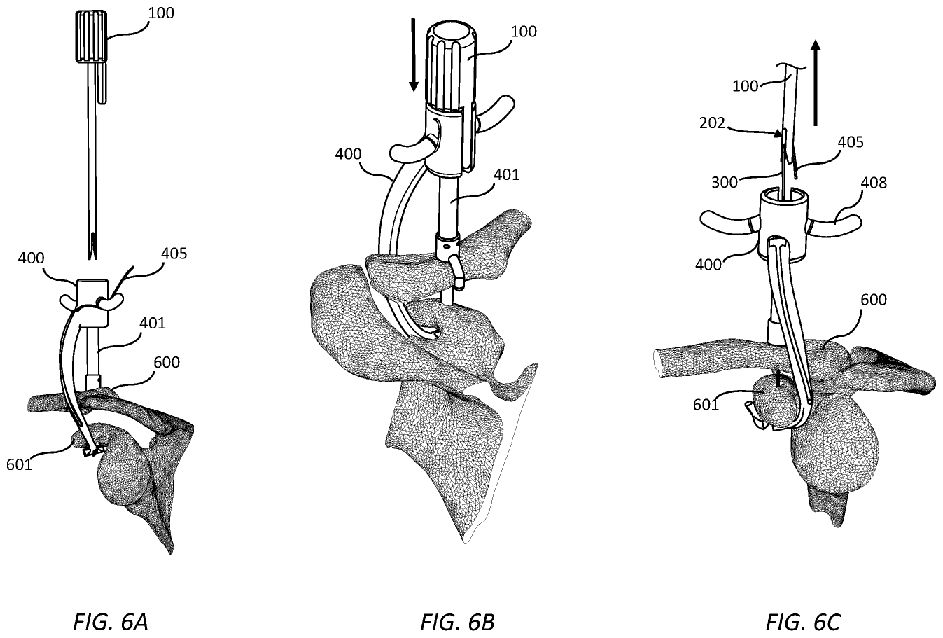

FIGS. 6A-6C illustrate exemplary steps of employing suture transporting device 100 to retrieve a shuttle suture through prepared bone apertures formed in a clavicle 600 and a coracoid 601, according to an embodiment of the present invention. In this example, FIG. 6A. shows drill guide system 400 positioned spanning the bony structures. An aperture in the bone has been formed by passing a drill through guide sleeve 401 and both bones. Shuttle cable transporting device 100 aligned for passing through guide sleeve 401 as described in FIGS. 5A-5C. FIG. 6B illustrates the advancement of shuttle cable transporting device 100 through guide sleeve 401 with orienting arm 104 engaging in orienting slot 407 to maintain the proper alignment and enable the capture of portion 406 (not shown) as described heretofore. As shown in FIG. 6C, end 405 is released from arm 408, enabling shuttle cable 300 to be retrieved through the apertures in clavicle 600 and coracoid 601 and exiting the proximal end of drill guide system 400 by retracting shuttle cable transporting device 100 from the assembly and allowing shuttle cable 300 freedom to slide through cable containment aperture 202.

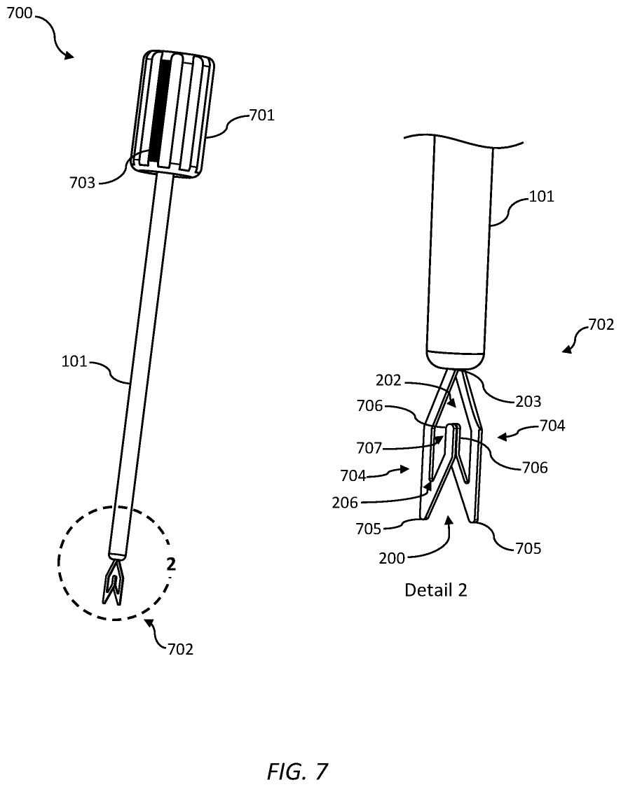

The discussion is now turned to other embodiments of the invention. FIG. 7A illustrates perspective and detail views of a shuttle cable transporting device 700 comprised of shaft 101, a handle 701 connected to the proximal end of shaft 101, and a capture mechanism 702 connected to the distal end of shaft 101. In this example, handle 701 includes a reference mark 703. Orienting mark 908 is formed at a position to function as a visual indication to enable the user orient capture mechanism 702 to the shuttle suture in lieu of the mechanical alignment method previously described. Capture mechanism 702 is formed having a first and second hook 704 oriented in a mirrored configuration forming mouth 200 between a set of bends 705. The shanks of hooks 704 are connected to the distal end of shaft 101 forming hinge 203. Each hook 704 has a point 706 which are overlap to form a and enclosing suture containment aperture 202 with the bite of each hook 704 forming saddle 206.

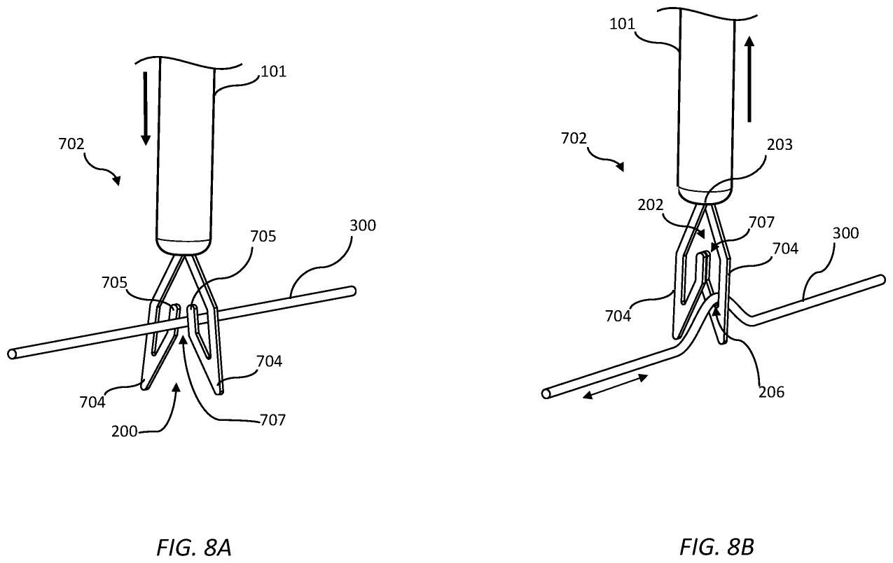

FIGS. 8A-8B illustrate exemplary steps for employing capture mechanism 702 to couple with a flexible member, in accordance with the disclosure. FIG. 8A illustrates shuttle cable 300 aligned with mouth 200 generally perpendicular to and between bends 705. As shaft 101 is translated distally, shuttle cable 300 causes gate 707 to open by forcing bends 705 to deflect and separate. FIG. 8B illustrates gate 707 closing after shuttle cable 300 advances proximally past gate 707. Hinge 203 resiliently return hooks 704 to their original positions, closing gate 707, and capturing shuttle cable 300 in cable containment aperture 202. After gate 707 returns to the closed position, shaft 101 is then retracted causing shuttle cable 300 to be retained in the throat of either hook 704 and may axially slide through cable containment aperture 202 on saddle 206.

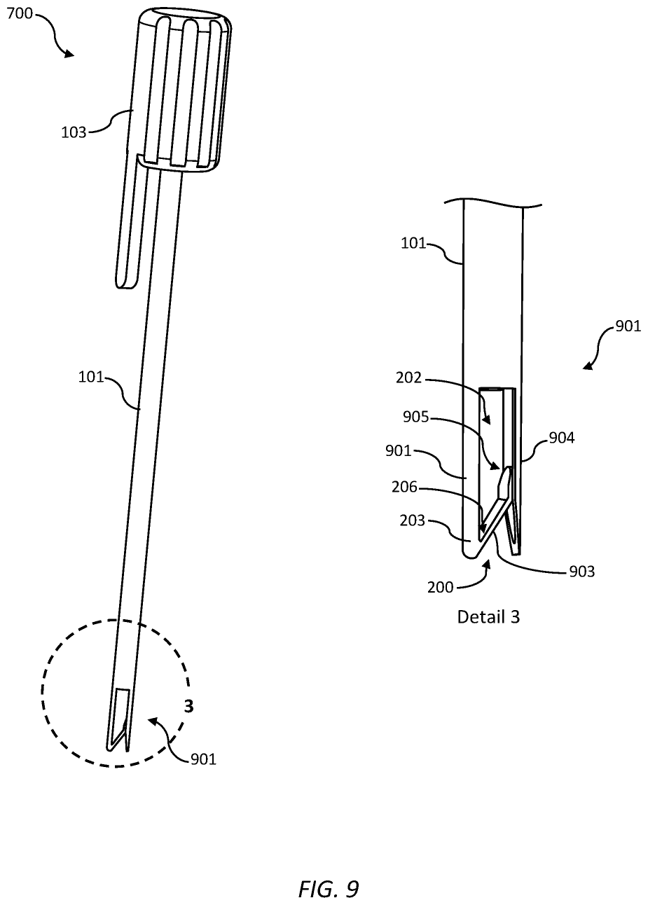

FIG. 9 illustrates perspective and detail views of a shuttle cable transporting device 900 comprised of shaft 101, handle 103 connected to the proximal end of shaft 101, and a capture mechanism 901 connected to the distal end of shaft 101, according to an embodiment of the present invention. In this example, capture mechanism 901 comprises a gate 905 formed by the tip of a hook 902 opposing a jaw 904 with mouth 200 formed between the end of jaw 904 and hinge 203. Bend 903 extends proximally from hinge 203, spanning mouth 200 at an angle, and is enabled to open away from jaw 904 when a force is applied to the distal side of bend 903 toward shaft 101, and resiliently close when the opening force is released, allowing a shuttle suture access to cable containment aperture 202 and be retained. In this example saddle 206 is formed on the inside radius of hook 902 at hinge 203.

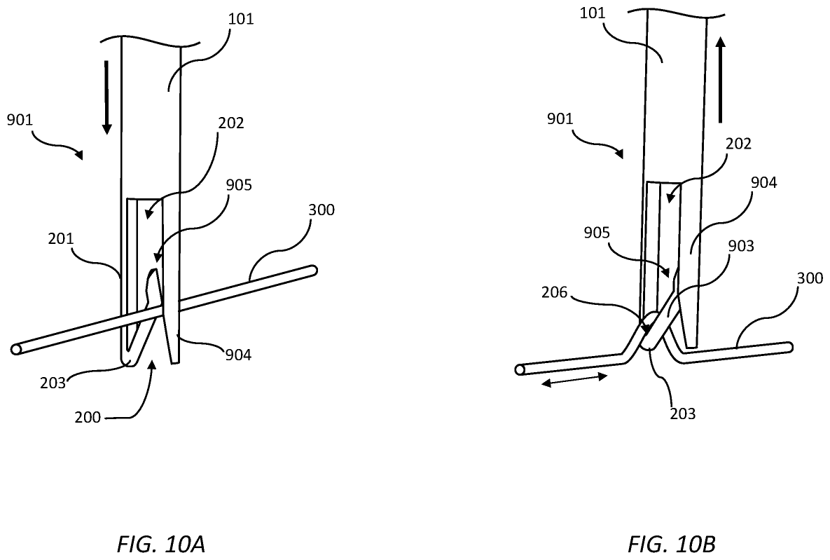

FIGS. 10A-10B illustrate exemplary steps for employing shuttle cable transporting device 900 to couple with a flexible member, in accordance with the disclosure. FIG. 10A illustrates shuttle cable 300 aligned with mouth 200 generally perpendicular to and between hinge 203 and the distal end of jaw 904. As shaft 101 is translated distally, shuttle cable 300 causes to gate 905 open and enter cable containment aperture 202. FIG. 8B illustrates gate 905 closed after shuttle cable 300 advances into cable containment aperture 202. Hinge 203 resiliently returns bend 903 to the original position contacting jaw 904. After gate 905 returns to the closed position, shaft 101 is then retracted causing shuttle cable 300 to be retained in the throat of hook 902 and may axially slide through cable containment aperture 202 on saddle 206.

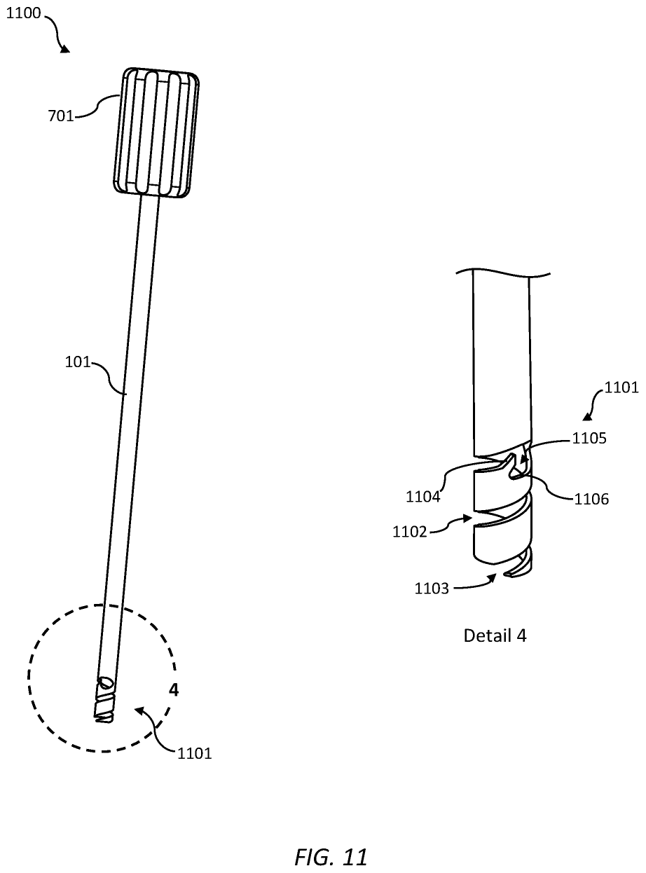

FIG. 11 illustrates perspective and detail views of a shuttle cable transporting device 1100, comprised of shaft 101, handle 701 connected to the proximal end of shaft 101, and a capture mechanism 1101 formed in the distal end of shaft 101, according to an embodiment of the present invention. In this example, capture mechanism 1101 is formed from a tubular body having longitudinal axis and a proximal end and a distal end therebetween. A helical channel 1102 is formed in shaft 101 with a mouth 1103 at the distal end allowing access for a shuttle suture to enter channel 1102 as shuttle cable transporting device 1100 is rotated about its longitudinal axis. At the proximal end of channel 1102 is a gate 1104 which reduces the width of channel 1102 to have a clearance or transition fit with the shuttle suture and to allow access to a containment aperture 1105. The distal portion of containment aperture 1105 forms a saddle 1106. It should be noted that several methods may be used to create channel 1102 such as forming a coil or creating a helical slot in a tubular body and should not limit the scope of the present invention.

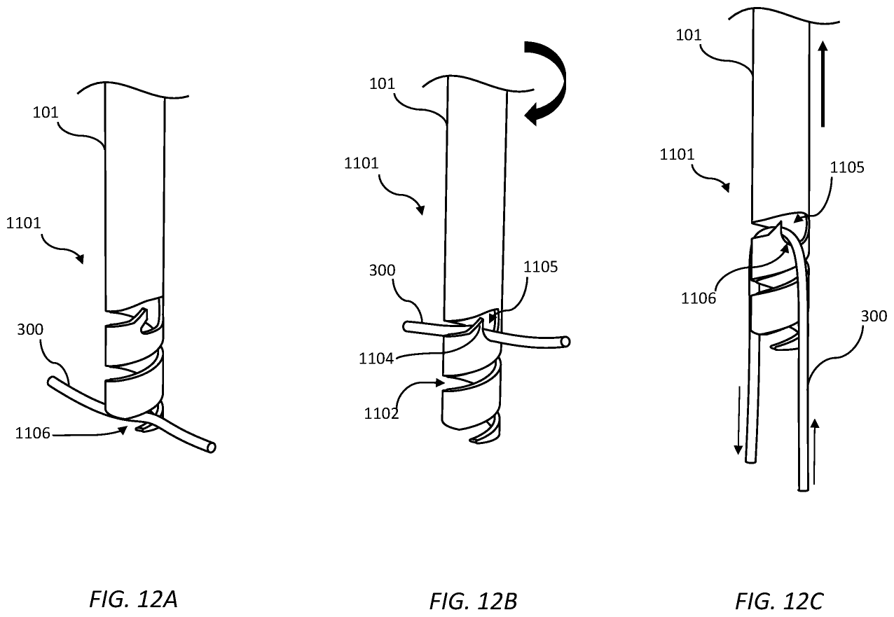

FIGS. 12A-12C illustrate exemplary steps for employing capture mechanism 1101 to couple with shuttle cable 300 and retain for transport, in accordance with the disclosure. FIG. 12A illustrates shuttle cable 300, generally perpendicular to the longitudinal axis of shaft 101, entering mouth 1103. A rotation of shaft 101 in the direction shown in FIG. 12B causes shuttle cable 300 to be drawn into channel 1102, translate proximally, and pass through gate 1104 to arrive in containment aperture 1105. As shown in FIG. 12C, shuttle cable 300 may axially slide through containment aperture 1105 on saddle 1106 as shaft 101 is translated proximally, thereby retaining shuttle cable 300 and enabling retrieval.

What is claimed is:

1. A surgical device for controlling a shuttle cable, comprising:
a shaft, comprising an elongate member having a longitudinal axis between a proximal end and a distal end; and
a capture mechanism formed at the distal end of said shaft, comprising:
a mouth, comprising a first jaw and a second jaw, the first and second jaws having cavity therebetween and accessible by a shuttle cable, the shuttle cable comprising a flexible member having a first end and a second end and a portion therebetween; and
a gate comprising at least one elongate body having a first end and a second end and a proximal side and a distal side, the first end connected to a jaw and in rigid communication with a flexible hinge, the hinge operable to allow the gate to deflect from a closed position to an open position by a force acting on the distal side of the gate, wherein the gate resiliently returns from an open position to a closed position in the absence of said opening force; and
a containment aperture, the aperture bounded by the proximal side of the gate, the first jaw, the second jaw, and the body of the capture mechanism and operable to allow the shuttle cable to slide axially therethrough;
wherein the open position of the gate allows passage of a portion of the shuttle cable into the containment aperture;
wherein the closed position of the gate separates the mouth from the containment aperture.

2. The surgical device of claim 1, further comprising a means for orienting the shaft to a feature on a second device about the longitudinal axis of the shaft, the means comprising at least one of the following: (a) a boss engaging a slot on the second device, (b) a slot engaging a boss on the second device, or (c) a feature formed on the device compared to a feature on a second device;
wherein the capture mechanism is operable for a shuttle cable coupled with the second device to pass through the gate when said features are aligned

3. A surgical device for controlling a shuttle cable, comprising:
a shaft, comprising an elongate member having a longitudinal axis between a proximal end and a distal end; and
a capture mechanism, comprising:
a tubular body having a longitudinal axis between a proximal end and a distal end, and a wall thickness between a lumen and an outer surface; and
a helical aperture formed radially from the lumen to the outer surface, the aperture connecting the distal surface to the proximal end of a containment aperture and operable for the passage of a shuttle cable, the shuttle cable comprising a flexible member having a first end and a second end and a portion therebetween; and
a containment aperture extending radially from the lumen to the outer surface and formed at the proximal end of the helical aperture;
wherein the containment aperture is operable to allow a shuttle cable to slide therethrough;
wherein rotation of the surgical device about the longitudinal axis causes the shuttle cable to enter the helical aperture;
wherein rotation of the suture transporting device causes a portion of the shuttle cable to translate proximally;
wherein rotation of the surgical device causes a portion of the shuttle cable to enter the containment aperture.

4. The surgical device of claim 3, wherein the capture mechanism includes a coil.

5. The surgical device of any of the previous claims further comprising a handle, wherein the handle is rigidly coupled to the proximal end of the shaft.

6. The surgical device of claim 5, the handle further comprising a plurality of gripping features, wherein the gripping features provide increased traction during grasping and manipulation of the transporting device during use.

7. The surgical device of claim 4, wherein the orienting means is connected to a handle.

8. A method of transporting a shuttle cable through tissue, comprising:
guiding the surgical device of any of the previous claims from a first side of a bone to a second side through an aperture formed therethrough;
aligning the capture mechanism of the surgical device with a portion of a shuttle cable, the shuttle cable comprising a first end and a second end and the portion therebetween, the portion operable for engagement with the capture mechanism, wherein a portion of the flexible member spans a portion of the bone aperture on the second side of the bone aperture;
coupling the surgical device to a portion of the shuttle cable; and
retrieving the capture mechanism through the bone aperture to the first side of the bone, wherein the flexible member is operable to slide through the containment aperture;
wherein the first end of the shuttle cable is available on the first side of the bone and the second end of the shuttle cable remains on the second side of the bone, and a portion of the shuttle cable is contained in the bone aperture.
